(19) United States
(12) Patent Application Publication
(10) Pub. No.: US 2021/0177796 A1
(43) Pub. Date: Jun. 17, 2021

(54) COMPOSITION FOR PREVENTING OR TREATING INFLAMMATORY DISEASES, CONTAINING MARINE FUNGUS PENICILLIUM SP. SF-5859-DERIVED CURVULARIN-TYPE METABOLITES

(71) Applicant: KOREA INSITITUTE OF OCEAN SCIENCE AND TECHNOLOGY, Busan (KR)

(72) Inventors: Joung Han YIM, Gyeonggi-do (KR); Il-Chan KIM, Gyeonggi-do (KR); Se Jong HAN, Gyeonggi-do (KR); Ui Joung YOUN, Incheon (KR); Hyun Cheol OH, Jeollabuk-do (KR); Youn-Chul KIM, Daejeon (KR); Jae Hak SOHN, Busan (KR); Minh Ha TRAN, Hanoi (VN); Wonmin KO, Busan (KR); Seung Jun LEE, Seoul (KR); Jae-Young SON, Busan (KR)

(21) Appl. No.: 16/759,281

(22) PCT Filed: Feb. 28, 2018
(86) PCT No.: PCT/KR2018/002498

§ 371 Date: Apr. 24, 2020

(30) Foreign Application Priority Data
Nov. 16, 2017 (KR) 10-2017-0152997

(51) Int. Cl. A61K 31/335; A61K 36/06; A23L 31/00

(52) U.S. Cl. CPC A61K 31/335; A23L 31/00; A61K 36/06

(57) ABSTRACT

The present invention relates to a pharmaceutical composition for preventing or treating inflammatory diseases and a food for preventing or alleviating inflammatory diseases, both of which contain curvularin-type metabolites as an active ingredient and, more specifically to a pharmaceutical composition for preventing or treating inflammatory diseases and a food for preventing or alleviating inflammatory diseases, both of which contain, as an active ingredient, marine fungus Penicillium sp. SF-5859-derived curvularin-type metabolites. A pharmaceutical composition, according to the present invention, for preventing or treating inflammatory diseases, containing curvularin-type metabolites derived from marine fungus Penicillium sp. SF-5859 (KCTC 13354 BP) inhibits the production of proinflammatory cytokines and mediators, thereby being effectively usable for the prevention or treatment of inflammatory diseases.

### TECHNICAL FIELD

The present invention relates to a pharmaceutical composition for preventing or treating inflammatory diseases, and a food for preventing or alleviating inflammatory diseases, which comprise a curvularin-type metabolite as an active ingredient, and more particularly to a pharmaceutical composition for preventing or treating inflammatory diseases, and a food for preventing or alleviating inflammatory diseases which comprise a curvularin-type metabolite derived from the marine fungus Penicillium sp. SF-5859 as an active ingredient.

### BACKGROUND ART

Inflammation is a vital part of the body's immune response and a useful defensive response to injury or damage, and is designed to mitigate the harmful effects of damage (Zhang, G et al., 2001. J. Clin. Invest, 107, 13-19). However, excess acute or chronic inflammation may cause serious disorders such as arthritis, asthma, colitis, Parkinson's disease, Alzheimer's disease, or sepsis (K. Lucas et al., 2013, Mol. Neurobiol. 48, 190-204). In the treatment of these diseases, the control of inflammation is essential.

The envelopes of Gram-negative bacteria, known as lipopolysaccharides (LPS), are powerful pro-inflammatory endotoxins. In macrophages and microglia, LPS stimulation may induce the production of pro-inflammatory mediators, including inducible nitric oxide synthase (iNOS)-derived nitric oxide (NO) and cyclooxygenase-2 (COX-2)-derived prostaglandin E2 (PGE₂). Nitric oxide (NO) is an inflammatory molecule produced by iNOS. An excessive increase in iNOS activity or production of nitric oxide is the etiology of various inflammatory diseases (McCartney-Francis et al., J. Exp. Med. 178, 749754, 1993; Szabo et al., New Horiz. 3, 232, 1995). PGE₂ synthesized by COX-2 is an important mediator of inflammatory symptoms such as fever and pain (Samuelsson et al., Pharmacol. Rev. 59:207224, 2007; Simmons et al., Pharmacol. Rev. 56:387437, 2004), and thus the inhibition of production of these inflammatory mediators is effective in the treatment of various inflammatory diseases. Accordingly, the inhibition of inflammatory cytokines and mediators may be a therapeutic target for the prevention of inflammation-related chronic diseases.

In addition, macrophages activated by LPS induce pro-inflammatory cytokines, including tumor necrosis factor-α (TNF-α), interleukin-1β (IL-1β), and interleukin-6 (IL-6) (T. Kawai et al., 2010, Nat. Immunol. 11, 373-384), and this is regulated by NF-κB. NF-κB consists of heterodimeric proteins of transcription factors p50 and p65. The heterodimeric domain interacts with the inhibitor IκBα, which inactivates NF-κB and retains a complex within the cytoplasm. The activity of the NF-κB system induced by LPS results in degradation of IκBα and migration of NF-κB to the nucleus. The transfer of NF-κB to the nucleus further induces the protein expression of iNOS and COX-2 and the mRNA expression of TNF-α and IL-1β.

The mitogen-activated protein kinase (MAPK) cascade activated in LPS-induced macrophages and microglia has been shown to play an essential role in inflammatory responses. There are three MAPK signaling pathways, namely c-Jun N-terminal kinase (JNK), extracellular signal-regulatory kinase (ERK), and P38 (M. Y. Peroval et al., 2013, PLoS ONE 8, e51243).

Meanwhile, marine microorganisms, including bacteria, cyanobacteria, microalgae, and fungi, are important sources of novel pharmacologically active secondary metabolites (Bugni and Ireland et al., Nat. Prod. Rep. 21:143163, 2004), and particularly, marine fungi are a rich and promising source of novel antiviral, anti-inflammatory, antibacterial, and anticancer agents (Bhadury et al., J. Ind. Microbiol. Biotechnol. 33:325337, 2006). Thus, for many years, studies on marine-derived fungi have disclosed new secondary metabolites and the pharmacological activity thereof.

Therefore, the inventors of the present invention isolated curvularin-type metabolites from the marine fungus Penicillium sp. SF-5859 and confirmed the anti-inflammatory effect of the compounds on inflammatory responses induced by LPS in RAW 264.7 macrophages, thus completing the present invention.

The above information described in the Background Art section is provided only for the purpose of improving understanding of the background of the present invention, and thus may not include information on the background art that is already known to those or ordinary skill in the art to which the present invention pertains.

### DISCLOSURE

### Technical Problem

It is an object of the present invention to provide a pharmaceutical composition for preventing or treating an inflammatory disease, and a food for preventing or alleviating an inflammatory disease, comprising a curvularin-type metabolite as an active ingredient.

### Technical Solution

To achieve the above objects, the present invention provides a pharmaceutical composition for preventing or treating an inflammatory disease comprising any one curvularin-type metabolite selected from the group consisting of compounds of Formula 1 to 4 below as an active ingredient:

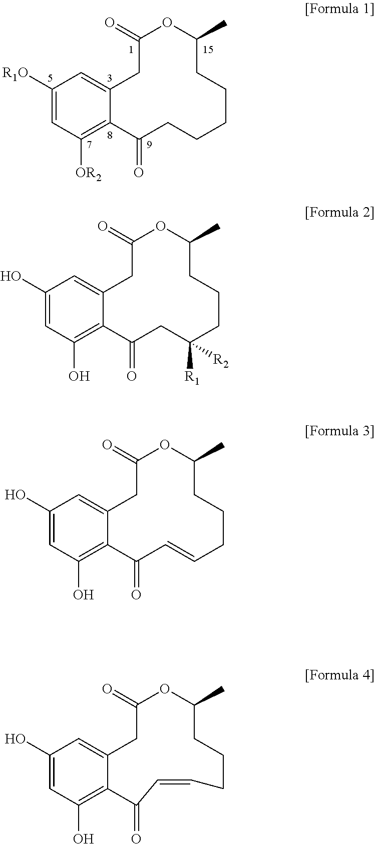

wherein R₁ and R₂ of Formula 1 are each independently H, OCH₃, or OAc, and R₁ and R₂ of Formula 2 are each independently H, OH, or OCH₃.

The present invention also provides a method of preventing or treating an inflammatory disease, comprising administering a pharmaceutically effective amount of any one curvularin-type metabolite selected from the group consisting of compounds of Formula 1 to 4.

The present invention also provides a use of any one curvularin-type metabolite selected from the group consisting of compounds of Formula 1 to 4 for preventing or treating an inflammatory disease.

The present invention also provides a use of any one curvularin-type metabolite selected from the group consisting of compounds of Formula 1 to 4 for manufacturing a drug for the prevention or treatment of an inflammatory disease.

The present invention also provides a food for preventing or alleviating an inflammatory disease comprising any one curvularin-type metabolite selected from the group consisting of compounds of Formula 1 to 4 as an active ingredient.

### DESCRIPTION OF DRAWINGS

FIG. 1 is a set of graphs showing the effects of a compound of Formula 3 on the protein expression levels of iNOS and COX-2 (a), and the mRNA expression levels of IL-1β, (b), IL-6 (c), and TNF-α (d) in RAW264.7 macrophages. Representative data or a mean value from three independent experiments were shown (*p<0.05 compared with the group treated with LPS).

FIG. 2 is a set of graphs showing the effects of the compound of Formula 3 on NF-κB activation (nuclear-p50 and nuclear-p65) (a), IκBα phosphorylation and degradation (b), and the DNA binding activity of NF-κB (c) in LPS-induced RAW264.7 macrophages. The data is expressed as a representative or mean value of three independent experiments (*p<0.05 compared with the group treated with LPS).

FIG. 3 is a set of graphs showing the effects of the compound of Formula 3 on p38 phosphorylation (a), JNK phosphorylation (b), and ERK phosphorylation (c) in LPS-induced RAW264.7 macrophages. Representative data from three independent experiments are shown.

### DETAILED DESCRIPTION AND EXEMPLARY EMBODIMENTS

Unless defined otherwise, all technical and scientific terms as used herein have the same meanings as commonly understood by one of ordinary skill in the art to which the present invention pertains. Generally, the nomenclature used herein is well known in the art and commonly used.

In one embodiment of the present invention, compounds of Formula 1 to 4, which are curvularin-type metabolites, were isolated from the marine-derived fungus Penicillium sp. SF-5859 (Accession No.: KCTC 13354BP) and the structures thereof were confirmed. In addition, it was confirmed that the curvularin-type metabolites (the compounds of Formula 1 to 4) inhibited the production of NO and PGE₂ in RAW264.7 macrophages and that the compound of Formula 3 inhibited the expression of iNOS and COX-2 and the mRNA expression of IL-1β, IL-6, and TNF-α, thereby exhibiting the effect of preventing or treating inflammatory diseases.

Therefore, in one aspect, the present invention relates to a pharmaceutical composition for preventing or treating an inflammatory disease comprising any one curvularin-type metabolite selected from the group consisting of compounds of Formula 1 to 4 below as an active ingredient:

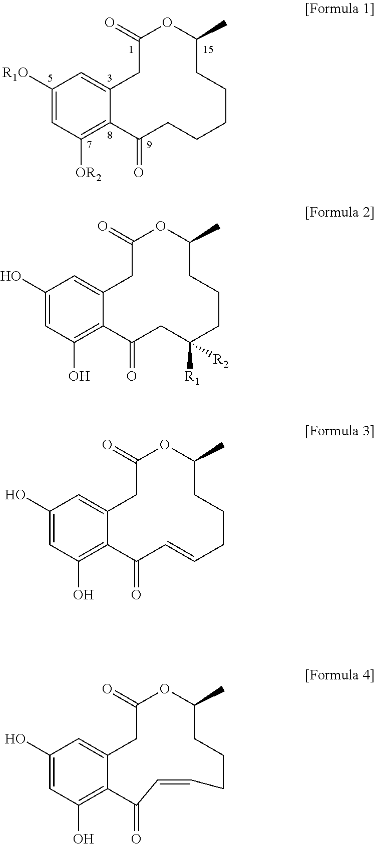

wherein R₁ and R₂ of Formula 1 are each independently H, OCH₃, or OAc, and R₁ and R₂ of Formula 2 are each independently H, OH, or OCH₃.

In another aspect, the present invention relates to a method of preventing or treating an inflammatory disease comprising administering a pharmaceutically effective amount of any one curvularin-type metabolite selected from the group consisting of compounds of Formula 1 to 4.

In another aspect, the present invention relates to a use of any one curvularin-type metabolite selected from the group consisting of compounds of Formula 1 to 4 for preventing or treating an inflammatory disease.

In another aspect, the present invention relates to a use of any one curvularin-type metabolite selected from the group consisting of compounds of Formula 1 to 4 for manufacturing a drug for the prevention or treatment of an inflammatory disease.

In the present invention, the metabolite of Formula 1 is defined as curvularin (Elzner, S.; et al., Inhibitors of inducible NO synthase expression: total synthesis of (S)-curvularin and its ring homologues. ChemMedChem 2008, 3, 924-939). The metabolite of Formula 1 may be a metabolite of any one selected from the group consisting of Formula 1a to 1e below:

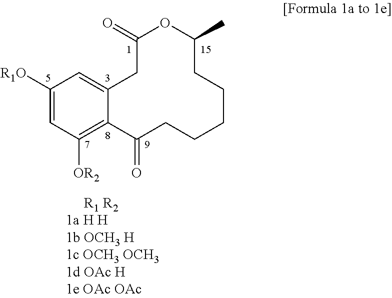

wherein R₁=H and R₂=H in Formula 1a, R₁=OCH₃ and R₂=H in Formula 1b, R₁=OCH₃ and R₂=OCH₃ in Formula 1c, R₁=OAc and R₂=H in Formula 1d, and R₁=OAc and R₂=OAc in Formula 1e.

The metabolite of Formula 2 may be a metabolite of any one selected from the group consisting of Formula 2a to 2d below:

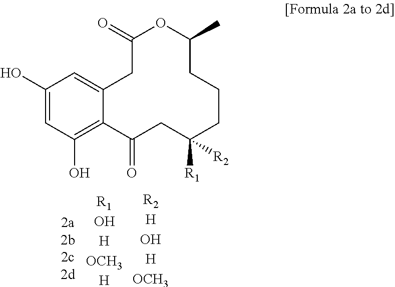

wherein, Formula 2a in which R₁=OH and R₂=H denotes (11R,15S)-11-hydroxycurvularin, Formula 2b in which R₁=H and R₂=OH denotes (11S,15S)-11-hydroxycurvularin (Greve, H.; et al., Apralactone A and a new stereochemical class of curvularins from the marine fungus Curvularia sp. Eur. J. Org. Chem. 2008, 2008, 5085-5092). Formula 2c in which R₁=OCH₃ and R₂=H denotes (11R,15S)-11-methoxycurvularin, and Formula 2d in which R₁=H and R₂=OCH₃ denotes (11S,15S)-11-methoxycurvularin (Liang, Q.; et al., First total syntheses and spectral data corrections of 11-α-methoxycurvularin and 11-β-methoxycurvularin. J. Org. Chem. 2007, 72, 9846-9849).

In addition, the metabolite of Formula 3 is defined as (10E,15S)-10,11-dehydrocurvularin (Greve, H.; et al., Apralactone A and a new stereochemical class of curvularins from the marine fungus Curvularia sp. Eur. J. Org. Chem. 2008, 2008, 5085-5092), and the metabolite of Formula 4 is defined as (10Z,15S)-10,11-dehydrocurvularin (Lai, S.; et al., Novel curvularin-type metabolites of a hybrid strain ME 0005 derived from Penicillium citreo-viride B. IFO 6200 and 4692. Tetrahedron Lett. 1989, 30, 2241-2244).

Curvularin-type metabolites are macrocyclic lactones produced by various fungi belonging to the genus Curvularia, the genus Penicillium, and the genus Alternaria, and are reported to have various physiological activities.

In the present invention, the curvularin-type metabolites may be isolated from the marine fungus Penicillium sp. SF-5859 (Accession No.: KCTC 13354BP).

In the present invention, the inflammatory disease may be selected from the group consisting of arthritis, rhinitis, hepatitis, keratitis, gastritis, enteritis, nephritis, bronchitis, pleurisy, peritonitis, spondylitis, pancreatitis, inflammatory pain, urethritis, cystitis, burn inflammation, dermatitis, periodontitis, gingivitis, and degenerative neuropathy, but the present invention is not limited thereto.

In the present invention, the pharmaceutical composition for preventing or treating an inflammatory disease may have one or more of the following characteristics:

1) Inhibition of the production of nitric oxide (NO) and prostaglandin E₂ (PGE₂);

2) Inhibition of the expression of inducible nitric oxide synthase (iNOS) and cyclooxygenase-2 (COX-2);

3) Inhibition of the expression of IL-1β, IL-6, and TNF-α;

4) Inhibition of the phosphorylation and degradation of inhibitor kappa B-α (IκB-α); and

5) Inhibition of the activation of nuclear factor kappa B (NF-κB).

In the present invention, the pharmaceutical composition for preventing or treating an inflammatory disease may have the characteristic of inhibiting the production of nitric oxide (NO) and prostaglandin E2 (PGE₂). In one embodiment of the present invention, it was confirmed that the compounds of Formula 1 to 4 inhibited the excess production of NO and PGE₂ (see Table 1). NO is a small molecule that is an intracellular mediator produced by various immune cells and plays a pivotal role in the physiological and pathological conditions of inflammatory symptoms. In addition, PGE₂ is able to modulate immune and inflammatory responses.

In the present invention, the pharmaceutical composition for preventing or treating an inflammatory disease may have the characteristic of inhibiting the expression of inducible nitric oxide synthase (iNOS) and cyclooxygenase-2 (COX-2).

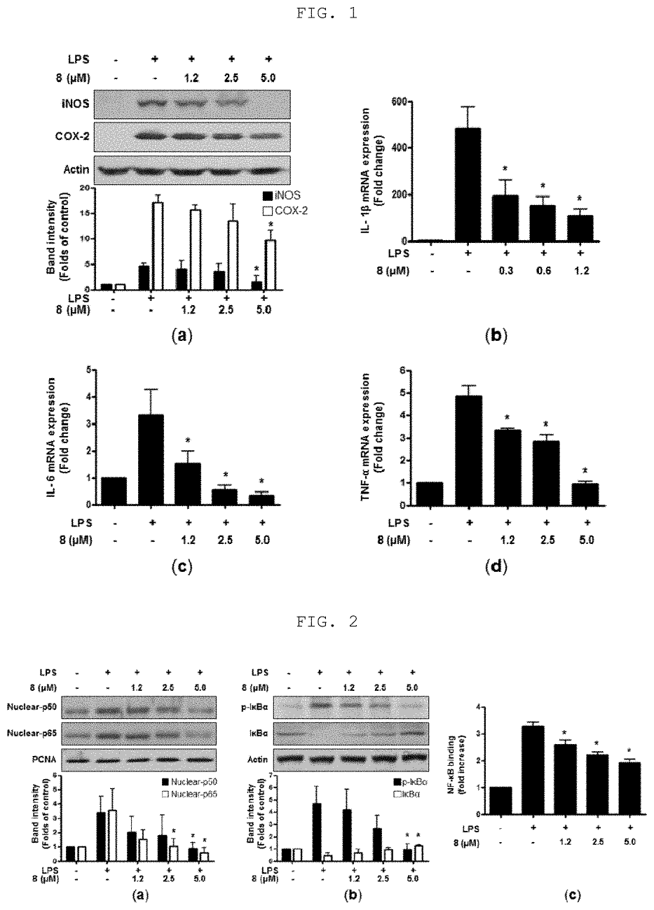

In one embodiment of the present invention, it was confirmed that the compound of Formula 3 reduced the excess protein expression of iNOS and COX-2 in LPS-induced cells in a dose-dependent manner (see FIG. 1(a)). iNOS and COX-2 are pro-inflammatory mediators, iNOS being an inflammatory molecule that produces NO, and COX-2 producing PGE₂. An excessive increase in the activity of iNOS and COX-2 may be the etiology of various inflammatory diseases.

In the present invention, the pharmaceutical composition for preventing or treating an inflammatory disease may have the characteristic of inhibiting the expression of IL-1β, IL-6, and TNF-α.

In one embodiment of the present invention, it was confirmed that, in LPS-induced cells, the compound of Formula 3 significantly inhibited the mRNA expression of IL-1β, IL-6, and TNF-α in a dose-dependent manner (see FIGS. 1(b) to 1(d)). These results indicated that the compound of Formula 3 attenuated the gene expression of pro-inflammatory cytokines at the transcriptional level. Excess production of pro-inflammatory cytokines such as TNF-α and IL contributes to the development of inflammatory diseases.

In the present invention, the pharmaceutical composition for preventing or treating an inflammatory disease may have the characteristic of inhibiting the phosphorylation and degradation of inhibitor kappa B-α (IκB-α) and inhibiting the activation of nuclear factor kappa B (NF-κB).

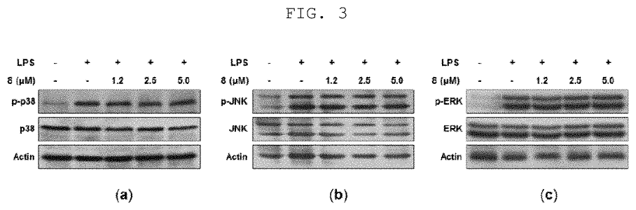

In one embodiment of the present invention, it was confirmed that (10E,15S)-10,11-dehydrocurvularin (compound of Formula 3), which is the most active metabolite, attenuated IκB-α phosphorylation and blocked the degradation of IκB-α in a concentration-dependent manner (see FIG. 2(b)). In addition, the compound of Formula 3 was able to suppress the induction of pro-inflammatory mediators and cytokines through down-regulation of the NF-κB signaling pathway.

It has been reported that many cellular signaling pathways and transcription factors are related to the expression of pro-inflammatory genes and enzymes in immune cells. NF-κB is an important transcription factor involved in inflammation-related diseases, and is known to modulate inflammatory genes the expression of and pro-inflammatory mediators such as iNOS and COX-2. In normal cells, NF-κB consists of inactive subunits of p50 and p65 bound to IκB-α. The NF-κB signaling pathway can be activated by LPS or other stimuli, which then phosphorylates IκB-α, leading to degradation and subsequent translocation of NF-κB into the nucleus.

In the present invention, the pharmaceutical composition for preventing or treating an inflammatory disease may not be mediated through the MAPK signaling pathway.

In one embodiment of the present invention, it was confirmed that the anti-inflammatory effect of the compound of Formula 3 was not mediated through the MAPK signaling pathway.

In one embodiment of the present invention, the pharmaceutical composition may further comprise a pharmaceutically acceptable carrier, an excipient, or a diluent.

Examples of suitable carriers, excipients, and diluents that may be included in the pharmaceutical composition comprise lactose, dextrose, sucrose, sorbitol, mannitol, xylitol, erythritol, maltitol, starch, acacia gum, alginates, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

In the present invention, the pharmaceutical composition may have, but is not limited to, any one formulation selected from the group consisting of powders, pills, granules, capsules, suspensions, liquids for internal use, emulsions, syrups, aqueous sterile solutions, non-aqueous solvents, freeze-dried preparations, and suppositories, according to a general method.

Formulations are prepared using commonly used diluents or excipients such as fillers, thickeners, binders, wetting agents, disintegrating agents, and surfactants. Solid preparations for oral administration include tablets, pills, powders, granules, capsules, and the like, and such a solid preparation is prepared by mixing the compound with at least one excipient, for example, starch, calcium carbonate, sucrose, lactose, or gelatin. In addition to simple excipients, lubricants such as magnesium stearate and talc are used. Liquid preparations for oral administration include suspensions, liquids for internal use, emulsions, syrups, and the like, and may include, in addition to commonly used simple dilutes such as water and liquid paraffin, various excipients, e.g., a wetting agent, a sweetener, a flavoring agent, and a preservative. Preparations for parenteral administration include aqueous sterile solutions, non-aqueous solvents, suspensions, emulsions, freeze-dried preparations, and suppositories. As the non-aqueous solvent and the suspension, propylene glycol, polyethylene glycol, vegetable oil such as olive oil, injectable esters such as ethyl oleate, and the like may be used. As suppository bases, Witepsol, Macrogol, Tween 60, cacao butter, laurin, glycerogelatin, and the like may be used.

In the present invention, the pharmaceutical composition may be administered orally or parenterally (e.g., intravenous, subcutaneous, intraperitoneal or topical application) depending on a desired method, and the dosage may vary depending on the state of health, body weight, age, and gender of patients, diet, excretion rate, the severity of disease, drug form, administration time, administration route, and administration period, but may be appropriately selected by one of ordinary skill in the art. However, for desired effects, the metabolite of the present invention may be administered at a dose of 0.001-1,000 mg/kg/day, preferably 0.01-100 mg/kg/day. The metabolite may be administered in a single dose or in multiple doses. The above dosage is not intended to limit the scope of the present invention in any way.

The pharmaceutical composition of the present invention may be used alone or in combination with surgery, radiotherapy, hormone treatment, chemotherapy, and methods using a biological response modifier, for the prevention or treatment of inflammatory diseases.

In another aspect, the present invention relates to a food for preventing or alleviating an inflammatory disease comprising any one curvularin-type metabolite selected from the group consisting of compounds of Formula 1 to 4 as an active ingredient.

In the present invention, the food for preventing or alleviating an inflammatory disease includes all forms such as nutritional supplements, health food, and food additives. The above types of food may be prepared into various forms according to general methods known in the art. For example, as a health food, the curvularin-type metabolites of the present invention may be prepared in the form of tea, juice, and drinks or may be ingested in granulated, capsulated, and powdered forms. In addition, the food may be prepared by adding the curvularin-type metabolite according to the present invention to beverages (including alcoholic beverages), fruits and processed foods thereof (e.g., canned fruits, bottled foods, jam, marmalade, and the like), fish, meat and processed foods thereof (e.g., ham, sausage, corn, beef, and the like), bread and noodles (e.g., Japanese-style noodles, buckwheat noodles, ramen, spaghetti, macaroni, and the like), fruit juices, various drinks, cookies, taffy, dairy products (e.g., butter, cheese, and the like), edible vegetable oils, margarine, vegetable proteins, retort foods, frozen foods, various seasonings (e.g., soybean paste, soy sauce, other sauces, and the like), and the like.

In the present invention, definitions of main terms used in the detailed description and the like are as follows.

As used herein, “inflammation,” which is one of biological tissue's defense responses to certain stimuli, refers to a bio-defense mechanism that attempts to recover the original condition by alleviating injuries caused by various harmful stimuli. Stimuli causing inflammation include infectious, chemical, and physical stimuli, and the inflammation process may be divided into acute inflammation and chronic inflammation. Acute inflammation is a short-term reaction that takes place within a few days, and plasma components, blood cells, or the like are involved in the removal of foreign substances via the microcirculatory system. Chronic inflammation has a long duration and appears as tissue proliferation or the like.

As used herein, “nitric oxide (NO)” is a substance in which the production amount thereof is increased by nitric oxide synthase when an inflammatory reaction is induced in a cell, and is a molecule that is an indicator of an inflammatory reaction. In the nervous system, nitric oxide is synthesized by nitric oxide synthase (NOS) of the nervous system present in neurons. The synthesized nitric oxide increases the production of cGMP in brain cells and, due to the increase, aids in the long-term storage of information taken in from the outside. NO, which is a free radical, is known to be involved in physiological and pathological processes. NO is synthesized through the oxidation of L-arginine by nitric oxide synthase (Atkan, et al., 75: 639-653, 2004).

As used herein, “COX-2” is an enzyme involved in producing prostaglandins, which are inflammatory response-related proteins, and an increase in the intracellular expression level of COX-2 may be an indicator of the progression of an inflammatory response.

As used herein, “prostaglandin E2 (PGE₂)” is an inflammatory mediator produced at an inflammatory site by COX-2, called prostaglandin endoperoxide synthase. PGE₂ is associated with many chronic inflammatory diseases, including cardiovascular diseases, arthritis, inflammatory bowel diseases, and chronic gastric ulcers (St-Onge, M. et al., Biochim. Biophys. Acta. 1771:1235-1245, 2007; Turini, M. E. et al., Annu. Rev. Med. 53:35-57, 2002; Rocca, B. et al., Int. Immunopharmacol. 2: 603-630, 2002; Singh, V. P. et al., Pharmacology 72:77-84, 2004).

As used herein, “MAPK” refers to a major signaling system that transduces a signal from the cell membrane to the nucleus when growth factors and the like activate receptors located on the cell membrane, thereby regulating the growth and differentiation of cells.

Hereinafter, the present invention will be described in further detail with reference to the following examples. These examples are provided for illustrative purposes only, and it will be obvious to those of ordinary skill in the art that these examples should not be construed as limiting the scope of the present invention.

### Example 1: General Experimental Procedures

Optical rotations were recorded using a Jasco P-2000 digital polarimeter (Jasco, Easton, Pa., USA). NMR spectra (1D and 2D) were recorded in a JEOL JNM ECP-400 spectrometer (400 MHz for ¹H, 100 MHz for ¹³C, JEOL Ltd., Akishima, Japan), and chemical shifts were referenced relative to the corresponding residual solvents signals (^{δ}H 2.05/^{δ}C 29.8 for acetone-d₆, ^{δ}H 7.26/^{δ}C 77.2 for CDCl3 and ^{δ}H 3.30/^{δ}C 49.0 for CD₃OD).

HMQC and HMBC experiments were optimized for ¹J_{CH}=140 Hz and ⁿJ_{CH}=8 Hz, respectively. HRESIMS data were obtained using an ESI Q-TOF MS/MS system (AB SCIEX Triple, SCIEX, Framingham, Mass., USA). Flash column chromatography was performed on silica gel (Kieselgel 60, 70-230 mesh and 230-400 mesh, Merck, Kenilworth, N.J., USA) and YMC octadecyl-functionalized silica gel (C₁₈, YMC CO., Kyoto, Japan). YMC semiprep-C₁₈ column (20×150 mm; 4 μm particle size; 80 Å pore size, 5 mL/min, YMC CO., Kyoto, Japan) and Shodex Ohpak SB 802.5 (8×300 mm; 6 μm particle size; 80 Å pore size, 0.6 mL/min, Showa Denko K.K., Tokyo, Japan) were used for HPLC (YoungLin, Anyang, Korea) separations. TLC was performed on Kieselgel 60 F254 (Merck, Kenilworth, N.J., USA) or reverse-phase (RP)-18 F254s (Merck, Kenilworth, N.J., USA) plates. Spots were visualized by spraying with 10% aqueous H₂SO₄ solution, followed by heating. All compounds were detected by UV absorption at 210 and 254 nm.

RPMI1640, fetal bovine serum (FBS), and other tissue culture reagents were purchased from Gibco BRL Co. (Grand Island, N.Y., USA). All other chemicals were obtained from Sigma-Aldrich Co. (St. Louis, Mo., USA). Primary antibodies (COX-2: sc-1745; iNOS: sc-650; IκB-α: sc-371; p-IκB-α: sc-8404; p50: sc-7178; p65: sc-8008, Santa Cruz Biotechnology, Dallas, Tex., USA, p-ERK: #9101; ERK: #9102; p-JNK: #9251; JNK: #9252S; p-p38: #9211; p38: 9212S, Cell Signaling Technology, Danvers, Mass., USA) and secondary antibodies (mouse: ap124p; goat: ap106p; rabbit: ap132p, Millipore, Billerica Mass., USA) were used. Enzyme-linked immunosorbent assay (ELISA) kits for PGE₂ were purchased from R and D Systems, Inc. (Minneapolis, Minn., USA).

### Example 2: Culture and Confirmation of Marine Fungus Penicillium sp. SF-5859

Penicillium sp. SF-5859 (Accession No.: KCTC 13354BP) was isolated from an unidentified sponge that was collected in the Ross Sea (76 06.25635 S 169 12.6752 E). The surface of the sponge was sterilized, and 1 g of the sample was ground with a mortar and pestle, followed by mixing with sterile seawater (10 mL). A portion (0.1 mL) of the sample was processed utilizing a spread plate method in potato dextrose agar (PDA) medium containing sterile seawater collected in the Busan area. The plate was incubated at 25° C. for 14 days. After subculturing the isolates several times, the pure cultures were selected and preserved at −70° C.

The fungal strain SF-5859 was identified based on the analysis of their rRNA sequences. A GenBank search with the 28S rRNA gene of SF-5859 (GenBank Accession No. KF745792) indicated Penicillium chrysogenum (FJ890400), P. steckii (HM469415), P. paxilli (FJ890408), and P. citrinum (JN938950), as the closest match showing sequence homology of 99.48%, 98.69%, 98.69%, and 98.43%, respectively. Therefore, the marine-derived fungal strain SF-5859 was characterized as Penicillium sp., but could not be definitively identified to a specific species.

### Example 3: Extraction and Isolation of Curvularin-Type Metabolites from Penicillium sp. SF-5859

The fungal strain Penicillium sp. SF-5859 was cultured on 10 g of Fernbach-style flasks each containing 100 g of semi-solid vermiculite and 400 mL of PDB with 3% (w/v) NaCl. The flasks were individually inoculated with 2 mL seed culture of the fungal strain and incubated at 25° C. for 14 days, then extracted with EtOAc (4 L per one flask). The combined extract solutions were filtered through filter paper and evaporated to dryness resulting in a crude extract SF5859 (2.2 g). The crude extract was fractionated on reversed phase (RP) C₁₈ flash column chromatography (5×30 cm), eluting with a stepwise gradient of 20, 40, 60, 80 and 100% (v/v) MeOH in H₂O (500 mL each) to give six fractions, i.e., SF5859-1 to SF5859-6, consecutively. The fraction SF5859-3 was applied to a chromatographic column packed with silica gel (2×30 cm). Subsequently, the column was eluted with gradients of CH₂Cl₂ in EtOAc (8/1 v/v, 200 mL) and (4/1 v/v, 150 mL) to yield a compound of Formula 3 (30.0 mg) and seven other fractions, SF5859-31 to SF5859-38. These were pooled based on TLC analysis.

The fourth fraction SF5859-34 was further purified by semi-preparative reverse-phase HPLC, eluting with a gradient of MeOH (60% to 80% in 20 min) in water (0.1% HCOOH) to afford a compound of Formula 4 (1.5 mg, t_{R}=13.5 min).

Similarly, the sixth fraction SF5859-36 was subjected to a semi-preparative RP HPLC column (50-80% MeOH in H₂O (0.1% HCOOH) over 30 min), giving two sub-fractions, SF5859-361 and SF5859-362, and a compound of Formula 2c (3.5 mg, t_{R}=46 min) was isolated from the sub-fraction SF5859-362 by performing on Shodex Ohpak SB 802.5 HPLC column (30-75% MeOH in H₂O over 50 min).

The seventh fraction SF5859-37 was separated into a compound of Formula 2b (1.5 mg, t_{R}=28 min) and two other sub-fractions using a semi-preparative RP HPLC column (30-60% MeOH in H₂O (0.1% HCOOH) in 30 min). Among these sub-fractions, SF5859-373 was further separated on a semi-preparative RP HPLC column (40-65% MeOH in H₂O (0.1% HCOOH) in 25 min) to thereby obtain a compound of Formula 2a (2.5 mg, t_{R}=20.5 min).

The eighth fraction SF5859-38 was separated firstly by a C₁₈ chromatographic column (1.5×20 cm), eluting with MeOH in H₂O (1/3 v/v), and the fraction SF5859-4 was chromatographed on a silica gel column (3×30 cm), eluting with CH₂Cl₂ in EtOAc (7/1 v/v). From this, a compound of Formula 1a (450.0 mg), which is a major metabolite was obtained, along with four other fractions.

The fifth fraction SF5859-45 was subjected to a final purification on a semi-preparative RP HPLC column (60-75% MeOH in H₂O (0.1% HCOOH) over 15 min) to afford a compound of Formula 2d (2 mg, t_{R}=13 min).

N,N-diisopropylethylamine (50 μL) was added to a solution of curvularin (Formula 1a, 15 mg) in 1 mL MeOH, followed by the addition of TMSCHN₂ (110 μL, 2.2 M in n-hexane). The reaction mixture was stirred for 15 hours at room temperature. The solution was then concentrated in a vacuo and extracted with EtOAc and H₂O prior to the evaporation of the organic phase. Subsequently, the residual material was subjected to semi-preparative RP HPLC eluting with a gradient of methanol in water (0.1% HCOOH) from 70% to 86% over 18 minutes to afford methylated products, i.e., compounds of Formula 1b (4 mg, t_{R}=14 min) and 1c (6 mg, t_{R}=16 min).

Curvularin (Formula 1a, 10 mg) was dissolved in 600 μL acetone, followed by the addition of acetic anhydride (600 μL). The reaction was started with adding a catalytic amount of N,N-dimethylpyridin-4-amine. The reaction mixture was stirred for 3 hours at room temperature. The resulting solution was dried in vacuo, and then partitioned with EtOAc and H₂O prior to the evaporation of the organic phase. Thereafter, the residual material was subjected to semi-preparative RP HPLC eluting with a gradient of methanol in water (0.1% HCOOH) from 62% to 80% over 19 minutes to afford the acetylated products, i.e., compounds of Formula 1d (2 mg, t_{R}=14 min) and 1e (3.5 mg, t_{R}=16 min).

### Example 4: Determination of Structures of Curvularin-Type Metabolites from Penicillium sp. SF-5859

4-1: Compounds of Formula 1b and 1c

5-O-methylcurvularin (1b): white amorphous powder; ¹H-NMR (CD₃OD, 400 MHz) and ¹³C-NMR data (CD₃OD, 100 MHz); HRESIMS m/z 309.1667 [M+H]⁺ (calcd. for C₁₇H₂₁D₂O₅ due to deuterium exchange, 309.1671).

5,7-Di-O-methylcurvularin (1c): white amorphous powder; 1H-NMR (CD₃OD, 400 MHz) and ¹³C-NMR data (CD₃OD, 100 MHz); HRESIMS m/z 321.1706 [M+H]⁺ (calcd. for C₁₈H₂₅O₅, 321.1702).

4-2: Compounds of Formula 1d and 1e

5-O-acetylcurvularin (1d): white amorphous powder; ¹H-NMR (acetone-d₆, 400 MHz); HRESIMS m/z 357.1336 [M+Na]⁺ (calcd. for C₁₈H₂₂NaO₆, 357.1314).

5,7-Di-O-acetylcurvularin (1e): white amorphous powder; ¹H-NMR (acetone-d₆, 400 MHz); HRESIMS m/z 399.1441 [M+Na]⁺ (calcd. for C₂₀H₂₄NaO₇, 399.1420).

4-3: Compound of Formula 4

It is noteworthy that, while the compound of Formula 4 has a negative specific rotation ([α]²²_{D}=−19.9 (c=0.15, EtOH), (10Z,15S*)-10,11-dehyrocurvularin that has the same planar structure as that of the compound of Formula 4 was isolated from a hybrid strain derived from Penicillium sp. with a positive specific rotation ([α]²²_{D}=+7.3 (c 0.78, EtOH); Lai, S et al., Novel curvularin-type metabolites of a hybrid strain ME 0005 derived from Penicillium citreo-viride B. IFO 6200 and 4692. Tetrahedron Lett. 1989, 30, 2241-2244). Considering the relationship between the sign of the specific rotation and the absolute configuration at C-15, it was suggested that the compound of Formula 4 is the first report of naturally-occurring (10Z,15S)-10,11-dehyrocurvularin, and the previously-reported (10Z,15S*)-10,11-dehyrocurvularin would have been an enantiomer of the compound of Formula 4.

### Example 5: Cell Culture and Cytotoxic Assay

In the present example, the cytotoxicity of each of the compounds of Formula 1 to 4 was confirmed in RAW264.7 macrophages by MTT assay.

RAW264.7 macrophages were maintained at a density of 5×10⁵ cells/mL in RPMI1640 medium supplemented with 10% heat-inactivated FBS, penicillin G (100 units/mL), streptomycin (100 mg/mL), and L-glutamine (2 mM), and were incubated at 37° C. in a humidified atmosphere containing 5% CO2. To determine cell viability, cells (1×10⁵ cells/well in 96-well plates) were incubated with 3-(4,5-dimethylthiazol-2-yl)-2,5-diphenyltetrazolium bromide (MTT) at a final concentration of 0.5 mg/mL for 3 hours, and the formazan formed was dissolved in acidic 2-propanol. The optical density was measured at 540 nm with a microplate reader (BioRad, Hercules, Calif., USA). The optical density of the formazan formed in control (untreated) cells was considered to represent 100% viability.

### Example 6: Effects of Curvularin-Type Metabolites from Penicillium sp. SF-5859 on Inhibiting Production of Nitrite and PGE₂

As an indicator of NO production in RAW264.7 macrophages, production of nitrite, which is a stable end-product of NO oxidation, was evaluated. The concentration of nitrite in conditioned media was determined based on the Griess reaction.

In the present example, RAW264.7 macrophages were pre-treated for 3 hours in the medium containing non-toxic concentrations (Table 1) of each compound (Formula 1 to 4), and then LPS (1 μg/mL) was treated for 24 hours. According to the LPS stimulation of RAW264.7 macrophages, the production of NO and PGE₂ was increased, and the effects of all compounds on the production levels of NO and PGE₂ were evaluated by the Griess reaction and a PGE₂ kit, respectively.

As a result, the compounds of Formula 1 to 4 inhibited the LPS-induced production of NO and PGE₂ in a dose-dependent manner, and the IC₅₀ values thereof are shown in Table 2. Based on comparison of the IC₅₀ values for compounds of Formula 1 to 4, it was evident that curvularin-type metabolites exhibited structure-dependent anti-inflammatory properties.

TABLE 1
Inhibitory effects of compounds of Formula 1 to 4 against
NO and PGE₂ production in LPS-treated RAW 264.7 macrophages

| | IC₅₀ (μM) | | |
|---|---|---|---|
| Compounds | NO | PGE₂ | Cytotoxicity (μM) ^{a} |
| 1a | 18.1 ± 5.2 | 18.7 ± 4.9 | 40 |
| 1b | >80 | 40.2 ± 5.1 | >80 |
| 1c | 60.6 ± 16.4 | 49.4 ± 14.0 | >80 |
| 1d | 46.9 ± 3.7 | 73.7 ± 17.1 | >80 |
| 1e | 77.5 ± 9.3 | >80 | >80 |
| 2a | 11.5 ± 2.7 | 15.6 ± 5.2 | 40 |
| 2b | 7.2 ± 1.6 | 14.1 ± 4.0 | 40 |
| 2c | 2.6 ± 0.4 | 3.0 ± 1.3 | 20 |
| 2d | 3.5 ± 0.5 | 6.0 ± 1.9 | 20 |
| 3 | 1.9 ± 0.3 | 2.7 ± 0.4 | 20 |
| 4 | 4.4 ± 0.8 | 6.2 ± 1.1 | 20 |

^{a} The maximum concentration not affecting cell viability.

### Example 7: Effects of Curvularin-Type Metabolites from Penicillium sp. SF-5859 on Expression of Pro-Inflammatory Enzymes and Pro-Inflammatory Cytokines

Among the curvularin-type metabolites of the present invention, the compound of Formula 3 was identified as the most active anti-inflammatory metabolite based on its IC₅₀ value (see Table 1).

Therefore, the inventors of the present invention further tested whether the inhibitory effects of the compound of Formula 3 against NO and PGE₂ productions are correlated with the protein expression of pro-inflammatory enzymes (e.g., iNOS or COX-2, respectively), which are known to catalyze the production of NO and PGEs in LPS-stimulated cells. RAW264.7 macrophages were pre-treated with the indicated concentrations of the compound of Formula 3 for 3 hours, and then stimulated with LPS for 24 hours. The presence of the compound of Formula 3 led to the attenuation of the excessive protein expression of iNOS and COX-2 in a dose-dependent manner (see FIG. 1(a)).

In addition, upon stimulation by LPS, macrophages can trigger the production of pro-inflammatory cytokines such as TNF-α and ILs. The overproduction of these cytokines contributes to the pathogenesis of inflammatory diseases. Thus, the inventors of the present invention further evaluated the effects of the compound of Formula 3 on the mRNA expression of pro-inflammatory cytokines in the LPS-induced cells. The cells were pre-treated with indicated concentration for 3 hours, followed by LPS stimulation (1 μg/mL) for 6 hours. The mRNA expression of pro-inflammatory cytokines was determined by RT-qPCR. As demonstrated in FIGS. 1(b) to 1(d), the compound of Formula 3 significantly suppressed the mRNA expression of IL-1β, IL-6, and TNF-α in a dose-dependent manner. These results indicated that the compound of Formula 3 attenuated the gene expression of pro-inflammatory cytokines at the transcriptional level.

### Example 8: Effect on IκB-α Phosphorylation and NF-κB Activity

It has been reported that many cellular signaling pathways and transcription factors are related to the expression of pro-inflammatory genes and enzymes in immune cells. Nuclear factor-κB (NF-κB) is an important transcription factor involved in inflammation-related disorders, and is known to modulate the inflammatory genes and the expression of pro-inflammatory mediators, such as iNOS and COX-2. In normal cells, NF-κB consists of inactive subunits of p50 and p65 bound to the inhibitor NF-κB (IκB-α). The NF-κB signaling pathway can be activated by LPS or other stimuli, which then phosphorylates IκB-α, leading to degradation and subsequent translocation of NF-κB into the nucleus.

In the present example, a NF-κB ELISA kit (Active Motif) was used to test the nuclear extracts and determine the degree of NF-κB binding. According to the data of the present example, pre-treatment with the compound of Formula 3 dose-dependently suppressed the nuclear translocation of p50 and p65 (see FIG. 2(a)). Furthermore, when the cells were treated with LPS alone, the phosphorylation level of IκB-α was increased, whereas the compound of Formula 3 attenuated this phosphorylation of IκB-α. In addition, the compound of Formula 3 blocked the degradation of IκB-α in a concentration-dependent manner (see FIG. 2(b)). In line with these, LPS-induced DNA binding activity of NF-κB was declined in the nuclear extracts of the cells co-treated with the compound of Formula 3 (see FIG. 2(c)). Taken together, it was suggested that the compound of Formula 3 could inhibit the induction of pro-inflammatory mediators and cytokines through the down-regulation of the NF-κB signaling pathway.

### Example 9: Effect on MAPK Pathway

Mitogen-activated protein kinase (MAPK) pathways are known to be involved in the expression of pro-inflammatory cytokines in macrophages. Thus, the effect of the compound of Formula 3 on the LPS-induced phosphorylation of MAPK was examined. Although the treatment of LPS for 30 minutes with the cells caused the phosphorylation of p38, c-Jun N-terminal kinase (JNK), and extracellular signal-regulated kinase (ERK), the data of the present example indicated that the compound of Formula 3 did not suppress the phosphorylation thereof (see FIGS. 3(a) to 3(c)). As a result, the anti-inflammatory effect of the compound of Formula 3 does not seem to be mediated through MAPK signaling pathways, and further study is needed in order to discover a specific target of the compound of Formula 3 involved in its anti-inflammatory activity.

### [Accession Number]

Depository Authority Name: Korea Research Institute of Bioscience and Biotechnology

Accession No.: KCTC13354BP

Accession date: Sep. 15, 2017

### INDUSTRIAL APPLICABILITY

According to the present invention, a pharmaceutical composition for preventing or treating inflammatory diseases comprising curvularin-type metabolites derived from marine fungus Penicillium sp. SF-5859 (KCTC 13354BP) inhibits the production of pro-inflammatory cytokines and mediators in RAW264.7 macrophages in relation to inflammation responses, and thus can be effectively used to prevent or treat inflammatory diseases.

While specific embodiments of the present invention have been described in detail, it will be obvious to those of ordinary skill in the art that these detailed descriptions are provided only to describe exemplary embodiments and these embodiments are not intended to limit the scope of the present invention. Thus, the substantial scope of the present invention should be defined by the appended claims and equivalents thereof.

1. A method of preventing or treating an inflammatory disease in a subject in need thereof, comprising administering a composition comprising any one curvularin-type metabolite selected from the group consisting of compounds of Formula 1 to 4 below as an active ingredient to the subject: 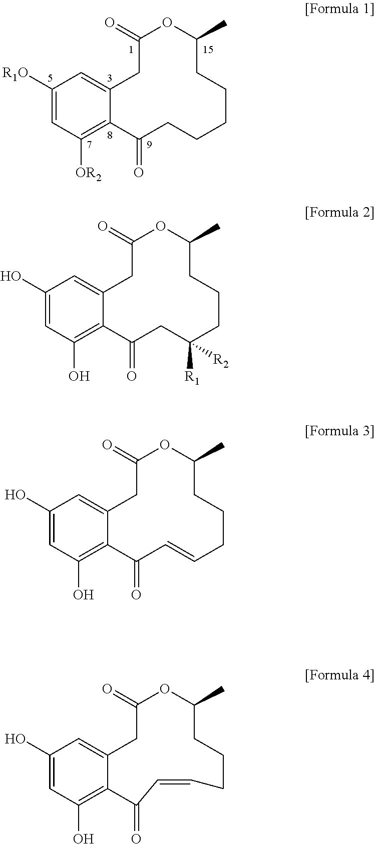
wherein R₁ and R₂ of Formula 1 are each independently H, OCH₃, or OAc, and R₁ and R₂ of Formula 2 are each independently H, OH, or OCH₃.

2. The method according to claim 1, wherein the curvularin-type metabolite is isolated from marine fungus Penicillium sp. SF-5859 (KCTC 13354BP).

3. The method according to claim 1, wherein the inflammatory disease is selected from the group consisting of arthritis, rhinitis, hepatitis, keratitis, gastritis, enteritis, nephritis, bronchitis, pleurisy, peritonitis, spondylitis, pancreatitis, inflammatory pain, urethritis, cystitis, burn inflammation, dermatitis, periodontitis, gingivitis, and degenerative neuropathy.

4. The method according to claim 1, wherein the composition has any one or more of the following characteristics:
1) Inhibition of the production of nitric oxide (NO) and prostaglandin E₂ (PGE₂);
2) Inhibition of the expression of inducible nitric oxide synthase (iNOS) and cyclooxygenase-2 (COX-2);
3) Inhibition of the expression of IL-1β, IL-6, and TNF-α;
4) Inhibition of the phosphorylation and degradation of inhibitor kappa B-α (IκB-α); and
5) Inhibition of the activation of nuclear factor kappa B (NF-κB).

5. The method according to claim 1, wherein the composition is a pharmaceutical composition and further comprises a pharmaceutically acceptable carrier, an excipient, or a diluent.

6. (canceled)

7. The method according to claim 1, wherein the composition is a food stuff or a dietary supplement.
